(19) United States
(12) Patent Application Publication
(10) Pub. No.: US 2021/0225493 A1
(43) Pub. Date: Jul. 22, 2021

(54) BODi (BORN ON FOR DEVICES & IMPLANTS) PROCESS FOR TRACKING THE QUALITY ASSURANCE OF SURGICAL DEVICES

(71) Applicant: KEITH J. PALMER, Frisco, TX (US)

(72) Inventor: KEITH J. PALMER, Frisco, TX (US)

(21) Appl. No.: 16/748,561

(22) Filed: Jan. 21, 2020

(51) Int. Cl. G16H 40/20; A61F 2/00

(52) U.S. Cl. CPC G16H 40/20; A61F 2250/0086; A61F 2/0095

(57) ABSTRACT

BODI is an implant prescription and supply chain revolutionizing service. BODI is bringing accountability to the industry for surgical implants. It lowers both costs and risks for implant manufacturers, distributors, healthcare providers, insurers, and ultimately patients. The purpose of BODI is to provide transparency and safety when handling surgical devices and ultimately reduce costs for members, surgeons, and insurers. Medwell's BODI reduces the need for error-prone data entry and provides a solution that is completely automated throughout the chain of custody and beyond. By implementing our BODI tracking system, hospitals, surgery, and manufacturers will recognize annual savings while meeting strict compliance regulations and improving patient outcomes. By 2025, the current database system will encompass “blockchain” technologic capabilities to further enhance the system for all users.

### CROSS-REFERENCE

Provisional patent application No. 62/804,635

### BACKGROUND OF THE INVENTION

### 1. Field of the Invention

The present invention relates generally to the process of tracking and handling of implantable surgical devices used in surgeries. More particularly, the invention relates to real-time tracking and quality assurance of surgical implants from manufacture to the moment devices are implanted in the patient's body.

### DESCRIPTION OF THE RELATED ART

For most medical implants, no clinical trials are undertaken. Instead, most medical 15 implants are approved on the basis of “substantial equivalence”. In other words, if a new implant is substantially equivalent to an implant already on the market, then it likely gets approved. The US Food & Drug Administration (FDA) requires manufacturers to submit a 510(k), which is a premarket submission made to FDA to demonstrate that the device to be marketed is at least as safe and effective, that is, substantially equivalent, to a legally 20 marketed device (21 CFR 807.92(a)(3)). This excludes the clinical trials, which US drugs are tested and approved upon-through clinical trials and placebos.

Example of similar concept being implemented is TrackCore Operating Room. TrackCore Operating Room is a tissue tracking and implant management software 5 eliminating the need for arduous manual processes. TrackCore does not provide time-stamped tracking of all the processes that surgical devices undergo such as washing, sterilization, radiation exposure, storage, and remaining life cycle of the devices.

The prior art has taught us that healthcare workers are required to ensure that all surgical instruments and implants are appropriately cleaned and sterilized according to the 10 nature of the item as instructed by the manufacturer. However, there is no standard process in place that handles the tracking of surgical implants at all times and research has shown that the repetitive exposure to radiation and the over sterilization of implants increase risks of complications to patients.

The prior art also teaches the importance of tracking surgical devices using a software 15 that records every step that implant undergoes with real time-updates incase a complication or a need for revision surgery occurs, we can easily determine the reason for such action. For instance, there was reporting of multiple adverse events occurring post orthopedic surgeries where surgical implants were used in the surgery. One case reported to the FDA (Food and Drug Administration) showed evidence of injury in patient due to rods breaking at both sides 20 of the patient's spine requiring removal of the implants and replacement through revision surgery.

There was no reason stated for the occurrence of the implant's breakage and unfortunately, the patient had to undergo spinal surgery for the second time after 5 years of the occurrence given the patient's age and health status.

Accordingly, BODI invention is an improvement over other products and technology because there is no product that tracks and records the cycles of sterilization for each 5 customized surgical implants. BODI offers that improvement and ensures that each surgical implant is customized to that specific case and patient and provides the exact history of that implant since it was manufactured.

### SUMMARY

BODI, a revolutionary tool developed by MedWell Consultants, LLC. dba MedWell 10 Solutions, tracks surgical implants from manufacture throughout use, reducing risk for hospitals, costs for manufacturers, and provides verification and accountability for medical device distributors.

The BODI system follows medical implants from the moment they enter circulation and through surgical procedures. The software acts as database system, tracking a medical 15 implant's cycle throughout its useful life, from the time manufactured, through procedures where it may be sterilized and re-sterilized, from the operating room to storage, and ultimately to use. By determining the amount of time that an implant has been under

radiation, we also reduce the risk to patients and cost to insurers of a less durable product.

BODI ensures an “implant integrity model” by collecting data and using safeguarded 20 procedures to identify why implants fail. The software process provides real time access to surgical implants shipment status, expiration dates and activity alerts, tracks implants by lot, serial, or QR codes in seconds, reduces waste, and offers a standardized process for handling all devices.

### BRIEF DESCRIPTION OF THE DRAWINGS

These and other features and advantages of the present invention will become more readily appreciated as the same becomes better understood by reference to the following detailed description when considered in connection with the accompanying drawings, wherein:

FIG. 1 is a simplified view showing the process of implant supply chain;

FIG. 2 is a fragmented view showing the process of implant supply chain in details.

FIG. 3.1 is the hospital/physician interface view on BODi—Prescription of required surgical devices

FIG. 3.2 is the hospital/physician requested order information—for tracking and updates.

FIG. 3.3 is the medical device manufacturers interface view on BODi—order request and shipping.

FIG. 3.4 is the case representatives interface view—confirmation of implants used during procedure.

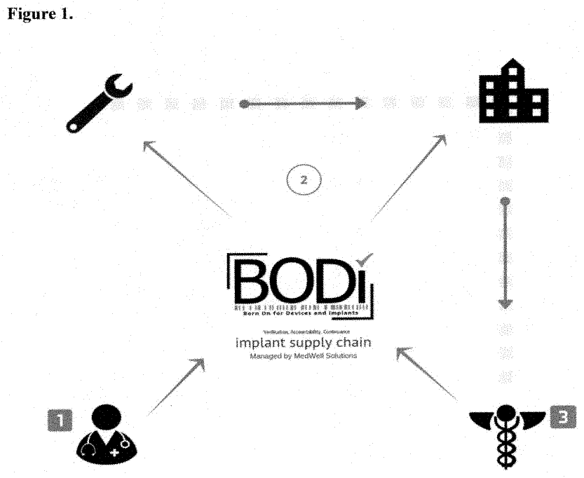

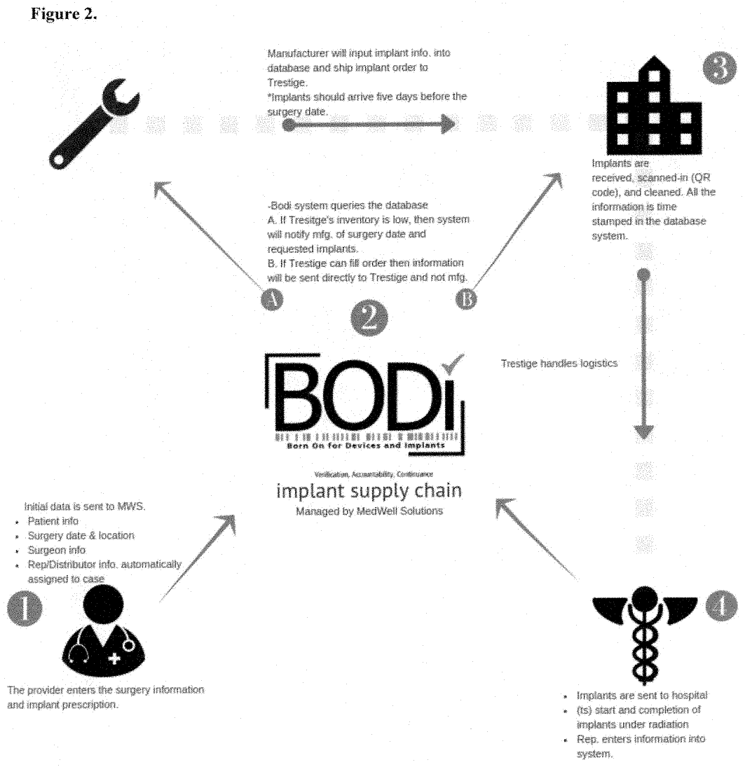

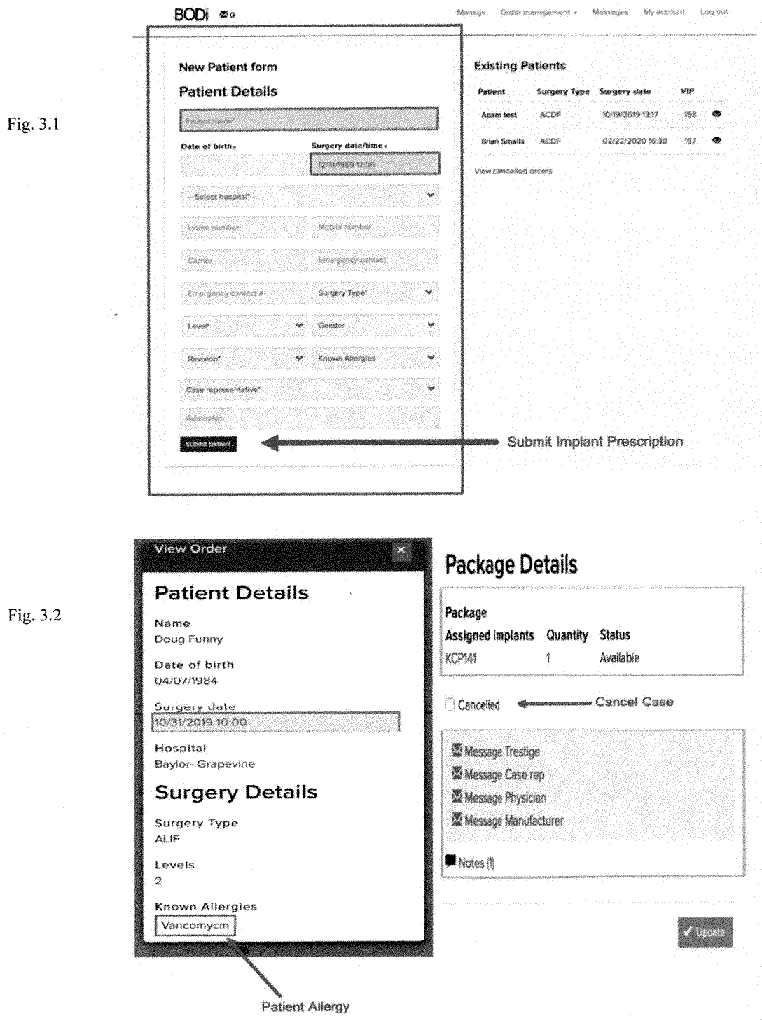

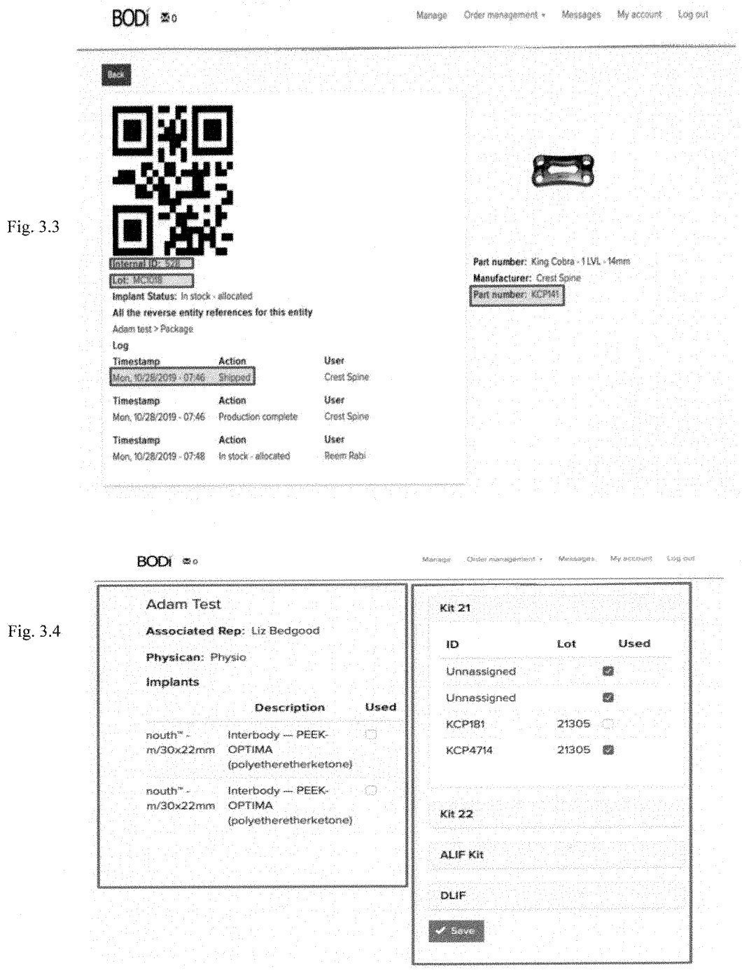

### DETAILED DESCRIPTION

Referring to the figures, wherein the numbers and letters represent the order and flow of each process in relation to the parties involved. The key members in this supply chain include; healthcare provider (physician or surgeon), MedWell Solutions employee, implant manufacturers (MFGs), Trestige (outsourced sterile processing/order fulfillment center), and surgical case representative.
Step one:
The provider enters the surgery information and implant prescription. The information is then sent to MedWell Solutions ‘BODI’ database.
Step two:
The system will query the inventory database of Trestige (Sterile processing center) to see if items are available before ordering from manufacture.
A. If inventory is low, then system will notify mfg. of surgery date and requested implants.
   Manufacturer will scan implant information into database and ship order to Trestige.
   Orders should arrive five days before the surgery date.
B. If order can be filled then information will be sent directly to Tresitge and not mfg.
Step three:
Orders are received and scanned in (time stamped). Trestige—sterile processing center—will then handle logistics
Step four:
Order arrives at hospital by delivery or pick-up by assigned representative. Representative will then time stamp start and completion of implants under radiation and sterilization cycles, then enter information into database, along with exact implants used during case if there were any additional outside of the original order kit.

By tracking the cycle of medical implants, BODI can manage risk in a unique way that drives collaboration between the healthcare providers, manufacturers, and insurers to safeguard patients. The system ensures a standardization process of cleaning, sterilization, and handling of these surgical implants. Each cycle of sterilization and radiation will be recorded and tracked via BODI technology. Once the standard limit of sterilization and radiation has been reached, the product will be eliminated, and a newly customizable surgical implant will replace it to ensure quality and safety of the surgical implant has been met. Currently, there is no existing system to determine the amount of time an implant has been under radiation, which may have a significant impact on the durability of the implant; higher amounts of radiation might equal higher risk for patients. A supply chain method of tracking a medical implant's cycle—from time manufactured, sterilized, re-sterilized, from the operating room to storage, etc.—can improve this system.

I claim:

1. A method including at least one feature depicted or described herein.
provide inventory list of all implants per procedure and ensure the system is updated with the correct information.
Information includes implant image, name, description, instructions for use (IFU), part #
Implant package only contains necessary implants for specific procedure and physician.

2. A computer readable medium including at least one feature depicted or described herein.
Name and contact information; Date the Device is was manufactured, shipped and provided.
QR code reader

3. An element including at least one feature depicted or described herein.

4. A system that complies with FDA Code of Federal Regulation (CFR) for medical implant devices.
CFR821.109 and CFR821.25
